(19) United States
(12) Patent Application Publication
(10) Pub. No.: US 2019/0019336 A1
(43) Pub. Date: Jan. 17, 2019

(54) Augmented Reality Biofeedback Display

(71) Applicant: Guy Coggins, Redwood City, CA (US)

(72) Inventor: Guy Coggins, Redwood City, CA (US)

(21) Appl. No.: 15/954,263

(22) Filed: Apr. 16, 2018

Related U.S. Application Data
(62) Division of application No. 14/432,177, filed on Mar. 27, 2015, now abandoned.

(51) Int. Cl. G06T 19/00; G06F 3/16; A61B 5/00; A61B 5/01; A61B 5/0205; A61B 5/0476; A61B 5/053; A61B 5/145

(52) U.S. Cl. CPC G06T 19/006; G06F 3/16; A61B 5/744; A61B 5/01; A61B 5/0205; A61B 5/14551; A61B 5/053; A61B 5/0533; A61B 5/14546; A61B 5/0476

(57) ABSTRACT

A system and method where at least one user's biofeedback information is captured or recorded using biofeedback devices, while the information of their physical properties are captured or recorded using at least one camera. Both sets of information is sent to computers to be processed into at least one information stream in a style of the choice of the user. The information stream(s) are then outputted to at least one device which they can be consumed by the user, such as (but not limited to) viewed on two-dimensional or three-dimensional screens, televisions and monitors, viewed through virtual/augmented reality devices, viewed on mobile devices, printed with printers, created with three-dimensional model printers, created using product printing services, saved on the Cloud, uploaded to websites/blogs and shown using image projectors. This combined visual information stream will usually take the form of (among other things) a live video or still image showing the user(s) with their biofeedback information interpreted as colors surrounding them, painted onto a model of the user(s), as colors projected onto the user(s) themselves, or as a color field appearing to surround or project from the user's body or musical instrument.

### CROSS-REFERENCE TO RELATED APPLICATIONS

The present application is a divisional application of U.S. application Ser. No. 14/432,177, filed Mar. 27, 2015, which takes priority from PCT Application No. PCT/US13/65482, filed Oct. 17, 2013, which claims the benefit of U.S. Provisional Application No. 61/744,606, filed Oct. 1, 2012, which are incorporated herein by reference in its entirety.

### BACKGROUND OF THE INVENTION

### Field of the Invention

The present invention relates generally to augmented reality displays, and more specifically to augmented reality displays that incorporate biofeedback or sonic information.

### Background

Biofeedback visual and photographic technology has been around for over forty years. In that time however, all of the available devices can only project their visual data in 2D without stereo 3D. Furthermore, all of the available devices usually require the user to not move at all from in front of the video camera recording the video of the user. On top of that, the device used to capture the user's biofeedback data is a stationary box on which the user must leave his or her hand, which further ties the user to a stationary position. All in all, this limits users from viewing their visual biofeedback data outside of a basic, limited, stationary position. This method of capturing and sharing biofeedback data is beginning to become more of an archaic inconvenience for users.

Augmented reality technology provides a way to enhance a user's real-time view of a physical, real-world environment by introducing virtual elements into the real-world scene. It is highly useful and desirable to introduce virtual elements into a real-world scene that are based on biofeedback information from a person or animal in the real-world scene. Such virtual elements enhance communication by providing useful information, or enhance the quality of a musical performance by displaying visual elements based on biofeedback or sonic information received from the performer.

### SUMMARY OF THE INVENTION

An object of the present invention is to provide a system and method for displaying biofeedback-based information as augmented reality.

Another object of the present invention is to enhance a musical performance by displaying visual information based on the musical sound in an augmented reality system.

In an embodiment, the present invention is a system comprising a camera that continuously captures a real-world scene including a user, a biofeedback sensor that continuously measures a biological parameter of the user, a computer that processes the information provided by the biofeedback sensor into visual information, and detects the location of the user's body, and a display module that displays the real-world scene with the visual information overlaid on top, or around, the user's body. The display module can be a smartphone screen, a tablet screen, a computer screen, a television, a projector, a wearable display such as virtual-reality glasses, a 3D display, or any other device capable of displaying the visual information and the real-world scene. The display module can also project the visual information directly onto the user's body. The biofeedback sensor can measure any biological parameter, such as body temperature, skin conductance, galvanic resistance, brainwaves, heart rate and rate variation, muscle signals, brain waves, or blood pressure.

In another embodiment, the present invention comprises multiple biofeedback sensors that measure biological parameters of multiple users and display them to other users.

In another embodiment, the present invention is a system comprising a camera that continuously captures a real-world scene including a user or a musical instrument, a music sensor that continuously senses musical sound or musical information produced by the user or by the user's musical instrument, a computer that processes the information provided by the music sensor into visual information and detects the location of the user's body in the real-world scene, and a display module that displays the real world scene with the visual information overlaid on top, or around, the user's body. The display module can be a projection screen such as are used in live music performance, a wearable display such as virtual-reality glasses, a smartphone screen, a tablet screen, a computer screen, a television, a projector, a 3D display, or any other device capable of displaying the visual information and the real-world scene. The display module can also project the information directly onto the user's body. The visual information can be presented as a color field that appears to surround the user's body, musical instrument, or both.

In another embodiment of the present invention, the biofeedback sensor is a medical sensor designed to measure the level of a medication in a patient's bloodstream or some other medical parameter such as blood sugar level, blood oxygen level, pain levels, and so on. The medical parameter can then be displayed to a doctor or nurse as an “aura” around the patient, as text “attached” to the patient's body, or as animated images. The biofeedback sensor could also be used to measure the level of alcohol or other recreational drugs in the user's blood.

### BRIEF DESCRIPTION OF THE DRAWINGS

FIG. 1 is a typical single-user implementation of the present invention;

FIG. 2 is a multiple user, multiple connection implementation of the present invention;

FIG. 3 is a multiple 2D- and 3D-screen implementation of the present invention;

FIG. 4 is a multi-camera, multiple 3D viewer implementation of the present invention;

FIG. 5a is a mobile implementation of the present invention;

FIG. 5b is a detailed front view of the individual elements in the mobile implementation of FIG. 5a;

FIG. 5c is a back view of the individual elements in the mobile implementation of FIG. 5a;

FIG. 6 is a kiosk implementation of the present invention;

FIG. 7 is a multiple printer implementation of the present invention;

FIG. 8 is an internet/online implementation of the present invention;

FIG. 9 is a projection implementation of the present invention;

FIG. 10a is a live music concert implementation of the present invention;

FIG. 10b is a straight-on view of the projection element in the live music concert implementation of FIG. 10a.

### DETAILED DESCRIPTION OF THE INVENTION

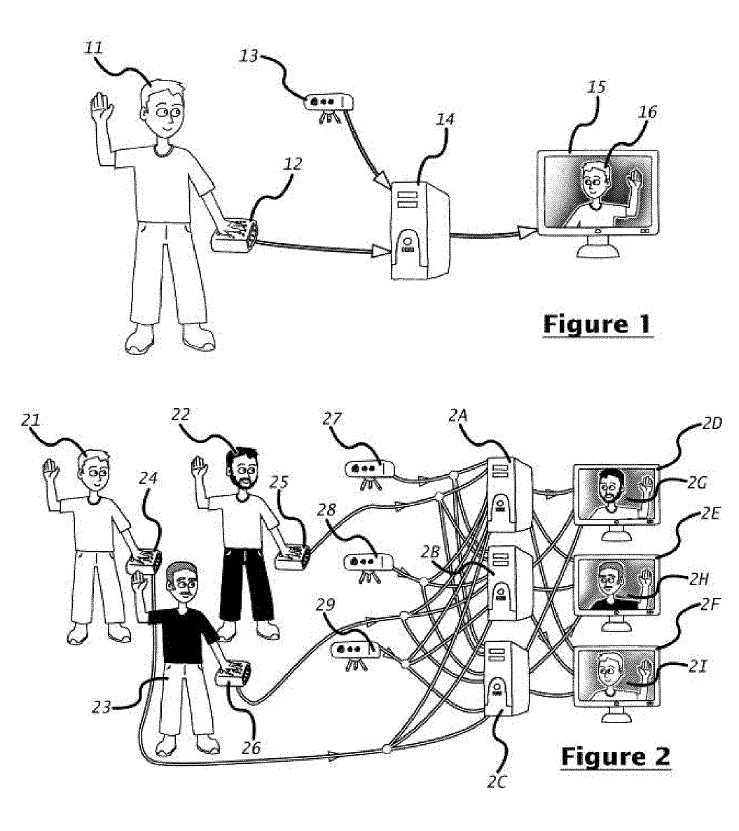

Referring now to the invention in more detail, in FIGS. 1 to 10, there is shown at least one user 11, 21, 22, 23, 31, 32, 41, 51, 61, 71, 81, 91, 101, 10H, whose biofeedback information is captured or recorded using biofeedback devices 12, 24, 25, 26, 33, 34, 42, 57, 62, 72, 82, 92 or other equipment that can be used to simulate biofeedback responses 102, 103, 104, while the information of their physical properties are captured or recorded using cameras 13, 27, 28, 29, 35, 43, 44, 53, 58, 63, 73, 83, 93, 107. Both sets of information is sent to computers 14, 2A, 2B, 2C, 36, 45, 55, 64, 74, 84, 94, 108, to be processed into at least one information stream in a style of the choice of the user 11, 21, 22, 23, 31, 32, 41, 51, 61, 71, 81, 91, 101, 10H. The information stream(s) are then sent to various devices which can be consumed by the user 11, 21, 22, 23, 31, 32, 41, 51, 61, 71, 81, 91, 101, 10H, such as (but not limited to) viewed on two-dimensional or three-dimensional screens, television and monitors 15, 2D, 2E, 2F, 37, 38, 46, 65, 109, viewed through virtual/augmented reality devices 47, viewed on mobile devices 52, 54, printed with printers 75, created with three-dimensional model printers 76, created using product printing services 77, saved on the Cloud 85, uploaded to websites/blogs 86, and shown using image projectors 95, or projected via a “Pepper's Ghost” system 10C, 10D, 101, 10J. This combined visual information stream will usually take the form of (among other things) a live video or still image showing the user or users 11, 21, 22, 23, 31, 32, 41, 51, 61, 71, 81, 91, 101, 10H with their biofeedback information interpreted as colors surrounding them 16, 2G, 2H, 21, 39, 3A, 48, 49, 4A, 56, 66, 78, 7A, 7B, 87, 10E, painted onto a model of the user or users 79, or as colors projected onto the user or users themselves 96 or towards the user or users themselves 10F, 10G, 10K, 10L.

In more detail, referring to the invention of FIG. 1, the user 11 will be attached to any sort of biofeedback device 12; it itself can be a stationary device or something that the user can “wear” (such as a glove, shoe, hat or other article of clothing) which moves along with the user. The physical properties of the user 11—their visual information itself, distance relative to other objects in the same room, etc—which isn't captured by the biofeedback device 12 will be captured by a camera 13. Both the biofeedback device 12 and the camera 13 will send their information to a computer 14, either as a live data stream or as a single “snapshot” of the user 11 at that specific moment in time. The computer will then take both data streams and process that in a specific way that the user 11 specifies. In FIG. 1, both data streams are combined into a single stream of visual information 16 and projected onto a computer monitor 15, allowing the user 11 to see both data streams in the way they specified.

Referring now to FIG. 2, multiple users 21, 22, 23 can be attached to at least one biofeedback device 24, 25, 26, which records their biofeedback data, while cameras 27, 28, 29 capture data about their physical properties. The data from the biofeedback devices 24, 25, 26 and cameras 27, 28, 29 can then all be sent to multiple computers 2A, 2B, 2C. The computers 2A, 2B, 2C can then process all six data streams from the biofeedback devices 24, 25, 26 and cameras 27, 28, 29, each in a unique way as the users 21, 22, 23 wish to assign to them. The computers 2A, 2B, 2C can then send their completed processed information to each of the three computer monitors 2D, 2E, 2F which allows the users 21, 22, 23 to view the different data streams 2G, 2H, 21 created by each of the computers 2A, 2B, 2C.

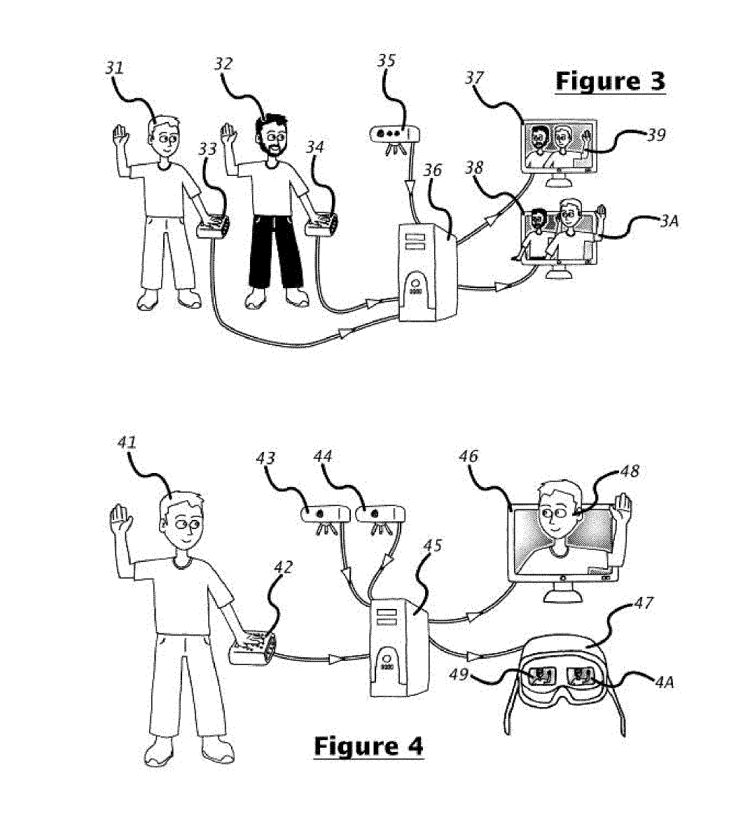

Referring now to FIG. 3, multiple users 31, 32 can be attached to at least one biofeedback device 33, 34, which records their biofeedback data, while a single camera 35 captures data about both of their physical properties. Both of these data streams are then sent to a computer 36 which is able to determine which biofeedback data stream from the two biofeedback devices 33, 34 belong to which of the two users 31, 32 recorded by the single camera 35. This information is then processed by the computer 36 in a manner determined by the users 31, 32, and the processed data stream is sent to two different monitors, a two-dimensional monitor 37 and a three-dimensional monitor 38, which can be viewed by the users 31, 32 in their respective formats (either as a 2D image 39 or as a 3D image 3A).

Referring now to FIG. 4, a user 41 is attached to at least one biofeedback device 42. The physical properties of the user 41 is recorded by two cameras 43, 44. Both data from the biofeedback device 42 and the cameras 43, 44 are sent to a computer 45. The computer 45 processes all three data streams in a manner determined by the user 41, and sent to two different devices capable of allowing the user 41 to view images as a three-dimensional image. The three-dimensional monitor 46 will project the combined data stream as a single 3D image 48. The virtual/augmented reality device 47 will allow the user 41 to interpret two separate data streams 49, 4A as a single 3D image.

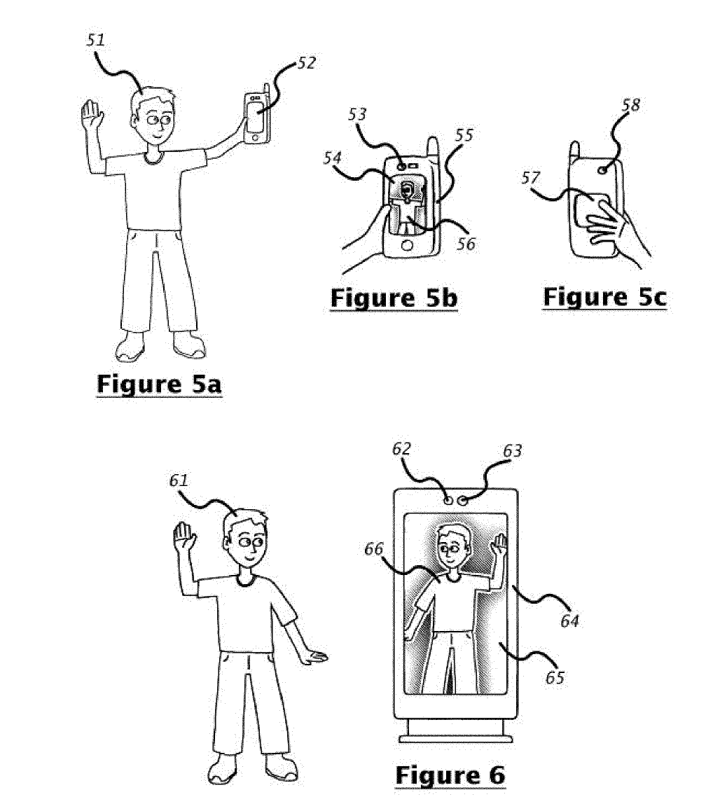

Referring now to FIG. 5a, a user 51 is holding a mobile device 52 in a certain way, which allows the mobile device 52 to process data in the manner of the present invention.

Referring now to FIG. 5c, which is the back view of the mobile device 52, there is at least one biofeedback device 57 that the user 51 can access. There is also another camera 58 which allows the user 51 to take a picture of themselves.

Referring now to FIG. 5b, which is a zoomed-in view of the mobile device 52, there is a camera 53 which can be used to capture data about the user 51. With the biofeedback data coming in from the biofeedback device 57 and visual data coming in from the camera(s) 53, 58, that data is then sent to the internal computer/processor 55 on the mobile device 52. The internal computer/processor 55 processes both data streams in a method which the user 51 chooses, and that data is then sent to the screen 54 of the mobile device 52. On the screen 54 is a visual representation 56 of both data streams from the biofeedback device 57 and the camera(s) 53, 58.

Referring now to FIG. 6, a user 61 walks in the vicinity of a freestanding, self-contained kiosk. The kiosk contains both a camera 62 and a form of biofeedback device 63 (which may or may not require physical contact from the user 61). The combined camera 62 and biofeedback device 63 data feed is sent to the internal computer 64 within the kiosk. The computer then processes the data of the two data feeds according to the settings provided either by the owner of the kiosk (which in that case cannot be changed by a user 61), or by the user 61 themselves through some manner via the kiosk's (touch)screen 65. Either way, the finalized processed data stream of the camera 62 and biofeedback device 63 is revealed on the screen 65 of the kiosk in the form of some kind of visual data 66. This data 66 can be seen live (as if the user 61 is in front of a mirror) or can be used to reveal certain kinds of advertisement according to the data gathered by the biofeedback device 63; either way, it is treated is something that can be “consumed” by the user 61.

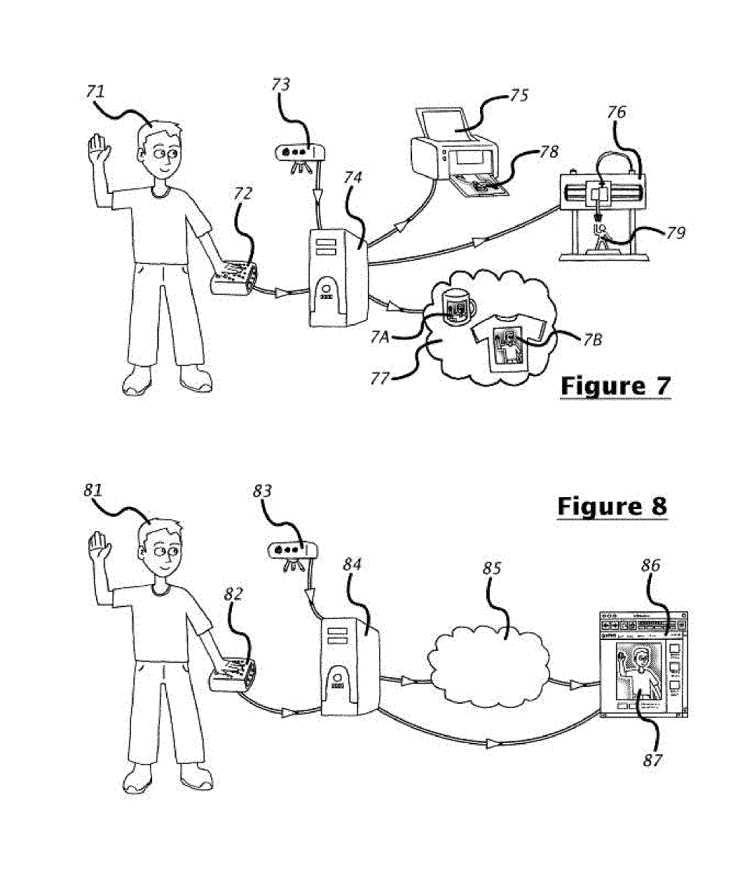

Referring now to FIG. 7, a user 71 is attached to at least one biofeedback device 72, and whose physical properties are captured by a camera 73. Both data streams from both devices are sent to a computer 74 to be processed in a manner according to the preferences of the user 71. This can include a printer 75 which prints out a snapshot 78 of the way the data from the biofeedback device 72 and the camera 73 is interpreted together, or a 3D model 79 created by a 3D printer 76, or as elements 7A, 7B on various merchandise which are created, stored and/or distributed by some form of merchandise creation system 77.

Referring now to FIG. 8, a user 81 is attached to at least one biofeedback device 82, and whose physical properties are captured by a camera 83. Both data streams from both devices are sent to a computer 84 to be processed in a manner according to the wishes of the user 81. The combined data stream 87 can then be uploaded to the Cloud 85, or immediately onto a website 86, or stored on the Cloud 85 for later updating to a website 86. The kind of data streams 87 that are saved and/or uploaded can include single still images or video backups of a session, or a live recording of a session for saving to video sites like YouTube, or as a live video feed through video-chatting services like Skype or Chat Roulette.

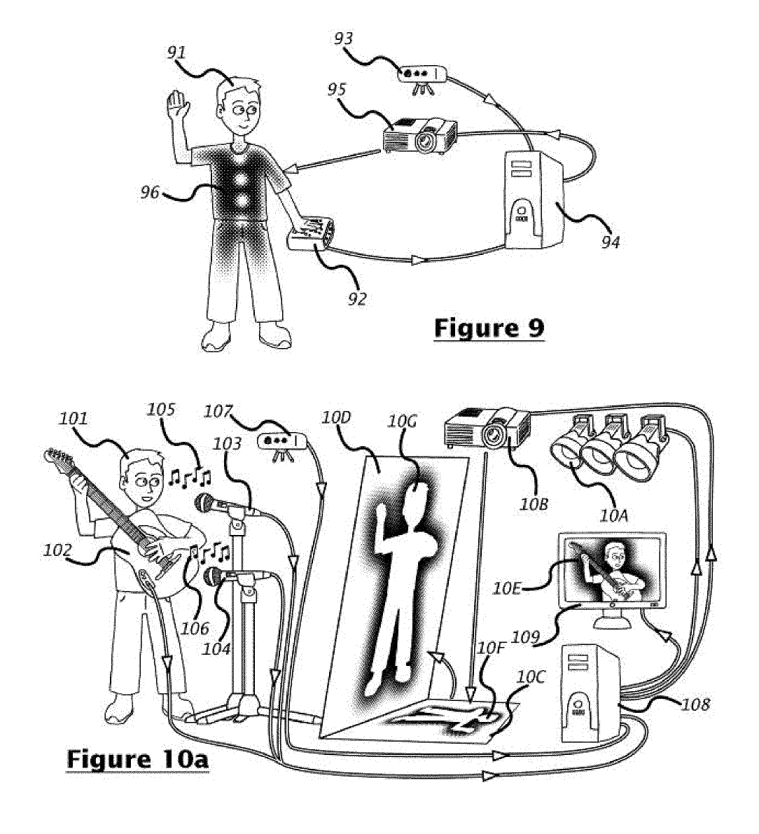

Referring now to FIG. 9, a user 91 is attached to at least one biofeedback device 92, and whose physical properties are captured by a camera 93. Both data streams from both devices are sent to a computer 94 to be processed in a manner according to the preferences of the user 91. The completed data stream can then be sent to a projector 95, which then can be projected 96 in any form onto a blank wall or the user 91 themselves. The camera 93 may then also record the projected image 96 over the user 91 and saved as a video file or single image onto a computer 94.

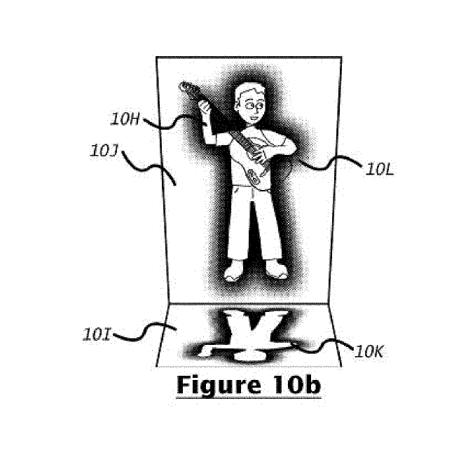

Referring now to FIG. 10a, a user 101 is in the presence of acoustic-related devices 102, 103, 104 which can interpret the user's sound producing capabilities as a biofeedback data stream. The user 101 may hold any musical instrument 102, and the sound of their voice 105 and/or musical instrument 106 is picked up by either a microphone 103, 104 or by the instrument 102 itself. Their physical properties are captured by a camera 107. Both sound/biofeedback and camera data streams are sent to a computer 108 to be processed in a manner according to the preferences of the user 101. The processed data stream can be sent either to a screen 109 in the form of some manner of visual data 10E, sent to a concert lighting system 10A, or sent to a projector 10B where the visual data 10F will be projected towards the user 101 via a “Pepper's Ghost”-style system 10C, 10D; specifically, the projector 10B will project its visual data 10F towards a reflector plate 10C, which will then reflect the visual data 10F projected onto it towards an adequately-sized, transparent sheet of glass 10D, which then will make the reflected visual data 10G appear to be in front of the user 101.

Referring now to FIG. 10b, the “Pepper's Ghost”-style system of reflective materials 101, 101 are shown at a straight-on view, showing how the reflected visual data 10K would be reflected off one reflective plate 101 towards the transparent reflective sheet of glass 101 such that the reflected image 10L would appear in front of the user 10H.

In further details, referring now to FIGS. 1-4 and FIG. 7-10, the user 11, 21, 22, 23, 31, 32, 41, 51, 61, 71, 81, 91, 101, 10H must be in regular contact with the biofeedback device 12, 24, 25, 26, 33, 34, 42, 72, 82, 92 or a comparable device 102, 103, 104 which can simulate biofeedback responses in order for the biofeedback data of the user 11, 21, 22, 23, 31, 32, 41, 51, 61, 71, 81, 91, 101, 10H to be properly recorded. However the user 11, 21, 22, 23, 31, 32, 41, 51, 61, 71, 81, 91, 101, 10H can be at any distance away from the camera 13, 27, 28, 29, 35, 43, 44, 73, 83, 93, 107, so long as the software on the computer 14, 2A, 2B, 2C, 36, 45, 74, 84, 94, 108 is able to adequately interpret the visual data from the camera 13, 27, 28, 29, 35, 43, 44, 73, 83, 93, 107 and recognize it as being from/of the user 11, 21, 22, 23, 31, 32, 41, 51, 61, 71, 81, 91, 101, 10H. However, the camera 13, 27, 28, 29, 35, 43, 44, 73, 83, 93, 107, biofeedback device 12, 24, 25, 26, 33, 34, 42, 72, 82, 92 (or its comparable device 102, 103, 104), computer 14, 2A, 2B, 2C, 36, 45, 74, 84, 94, 108, screen (and other visual devices) 15, 2D, 2E, 2F, 39, 3A, 46, 47, 95, 109, 10B, other lighting systems 10A and/or printers 75, 76, 77 may or may not be actually physically connected with one another or even in one another's physical presence such that the user 11, 21, 22, 23, 31, 32, 41, 51, 61, 71, 81, 91, 101, 10H has physical access to them; what matters is that there is a connection between each of the necessary systems.

Referring now to FIGS. 1-10, the camera 13, 27, 28, 29, 35, 43, 44, 53, 58, 62, 73, 83, 93, 107 can be of any resolution, just so long as the screen (and other visual devices) 15, 2D, 2E, 2F, 39, 3A, 46, 47, 54, 65, 95, 109, 10B, website 86 and/or printers 75, 76, 77 is capable of adequately rendering the visual data to the preference of the user 11, 21, 22, 23, 31, 32, 41, 51, 61, 71, 81, 91, 101, 10H. The camera 13, 27, 28, 29, 35, 43, 44, 53, 58, 62, 73, 83, 93, 107 can also be either a 2D, a “2D plus distance”, a 3D, a “3D plus distance” or any other camera that is capable of recording physical data about a user 11, 21, 22, 23, 31, 32, 41, 51, 61, 71, 81, 91, 101, 10H. Likewise, the screen (and other visual devices) 15, 2D, 2E, 2F, 39, 3A, 46, 47, 54, 65, 95, 109, 10B and/or printers 75, 76, 77 can be of any resolution or quality, so long as the user 11, 21, 22, 23, 31, 32, 41, 51, 61, 71, 81, 91, 101, 10H is able to adequately see their visual data to their preferences. The biofeedback devices 12, 24, 25, 26, 33, 34, 42, 57, 62, 72, 82, 92 (or its comparable device 102, 103, 104) also don't necessarily need to be in direct contact with the user 11, 21, 22, 23, 31, 32, 41, 51, 61, 71, 81, 91, 101, 10H; what matters is that the biofeedback devices 12, 24, 25, 26, 33, 34, 42, 57, 62, 72, 82, 92 (or its comparable device 102, 103, 104) are capable of recording the necessary physiological data of the user 11, 21, 22, 23, 31, 32, 41, 51, 61, 71, 81, 91, 101, 10H (which is the primary characteristic of “biofeedback devices”). The camera 13, 27, 28, 29, 35, 43, 44, 53, 58, 62, 73, 83, 93, 107 and biofeedback devices 12, 24, 25, 26, 33, 34, 42, 57, 62, 72, 82, 92 (or its comparable device 102, 103, 104) can also either passively capture their respective data and send that raw data to the computer(s) 14, 2A, 2B, 2C, 36, 45, 55, 64, 74, 84, 94, 108 for further processing, or they can process the data within themselves and send that processed data to the computer(s) 14, 2A, 2B, 2C, 36, 45, 55, 64, 74, 84, 94, 108 without requiring much (if any) further processing. Either way, the computers 14, 2A, 2B, 2C, 36, 45, 55, 64, 74, 84, 94, 108 must also be of sufficient processing capability as to handle at least two independent data streams from at least one biofeedback device(s) 12, 24, 25, 26, 33, 34, 42, 57, 62, 72, 82, 92 (or its comparable device 102, 103, 104) and at least one camera(s) 13, 27, 28, 29, 35, 43, 44, 53, 58, 63, 73, 83, 93, 107 (whether those data streams are raw, processed or otherwise), as well as exporting those two data streams-either as a single, combined stream, or simply forwarding the streams as is, or converting them to any other forms-to at least one of the following: two-dimensional or three-dimensional screens, television and monitors 15, 2D, 2E, 2F, 37, 38, 46, 65, 109, viewed through virtual/augmented reality devices 47, viewed on mobile devices 52, 54, printed with printers 75, created with three-dimensional model printers 76, created using product printing services 77, saved on the Cloud 85, uploaded to websites/blogs 86, and shown using image projectors 95, 10B or lighting systems 10A. The computers 14, 2A, 2B, 2C, 36, 45, 55, 64, 74, 84, 94, 108 may also have software on it that allows the user 11, 21, 22, 23, 31, 32, 41, 51, 61, 71, 81, 91, 101, 10H to save still images or video of the data feed 16, 2G, 2H, 21, 39, 3A, 48, 56, 66, 87, 96, 10E, 10G, 10L.

Referring now to FIG. 3, the three-dimensional monitor 38 can be of any form, such as (but not limited to) a monitor that requires specialty glasses in order to see the 3D image 3A, or a monitor that can be viewed without specialty glasses.

Referring now to FIG. 4, the cameras 43, 44 can be any distance away from one another, although if the intent is to create a 3D image 48, 49, 4A, then it is recommended that the two cameras not be too far apart (ideally the general distance between one's own eyes). The virtual/augmented reality device 47 can be of any form, including (but not limited to) actual “goggles” you have to wear, as a simple HUD display device (such as “Google Glasses”), or as software on a mobile or video game system (such as the “Nintendo 3DS”).

Referring now to FIG. 5a-5c, the user 51 must be in regular contact with the mobile device 52 and its biofeedback device 57. By virtue of that, the user 51 will also be in close proximity to the camera 53, 58 which will record the visual data of the user. The user 51 may change the distance between themselves and the mobile device 52, which won't affect the function of the invention. The mobile device 52 must also be of a type that has at least one camera (either front facing 53 or back facing 58), have some manner of biofeedback interaction 57—which may be one of the cameras 53, 58 or the phone's (touch)screen 54—and an internal computer/processor 55 which can handle live video and biofeedback data feeds as well as the processing of them into a single or multiple data feed 56. The mobile device 52 must also be able to accept the installation of software onto it, namely the software necessary to process both camera 53, 58 and biofeedback device 57 data feeds into a manner which the user 51 prefers. However, whether or not the mobile device 52 is capable of cellular or wi-fi communication is a non-issue; it should be able to do everything covered in this invention without the use of cellular or wi-fi communication.

Referring now to FIG. 6, the user 61 must be within a close enough range to the kiosk that would allow both the camera 62 and the biofeedback device 63 to capture data about the user 61. If either the camera 62 or the biofeedback device 63 is not capable of accurately capturing data about the user 61, then both will not work. The internal computer 64 must also have the capability to interpret both data feeds from the camera 62 and the biofeedback device 63 and either show the user 61 a visual interpretation of the combined data feeds 66, or show specific other imagery—advertising, commercials, text, etc—which are related to how both data feeds are interpreted. The kiosk must also be of a particular size in order for the attention of the user 61 to be caught by the kiosk and drawn to it to interact with it.

Referring now to FIG. 7, the printed materials 77, 78, 79, can be of any shape, size or quality. The printed merchandise 77 can be printed and sent to a user 71 immediately, or saved for later printing and/or purchase.

Referring now to FIG. 8, both the Cloud 85 and the website 86 (access to and from) must be of sufficient speed to handle a regular data feed sent by the computer 84. The final data stream 87 should also be in a graphics or video format which the Cloud 85 and/or the website 86 is capable of properly processing.

Referring now to FIG. 9, the projector 95 should be a proper distance away from the user 91 such that the image it projects 96 lines up where the user 91 feels it should. It is also likewise ideal for the projector 95 to be stationary as the camera 93 and the software on the computer 94 should be able to keep proper track of the user 91 without requiring the projector 95 to move to ensure that the image that it projects 96 remains projected onto the user. However, this does not prevent the user 91 from using a kind of projector 95 that is able to track the user 91 so that the user 91 never moves outside of the visual range of the projector 95.

Referring now to FIG. 10a, the user 101 can use any musical instrument 102 they wish, or they could not even use one at all. The core idea is that the user's current physiological state, as in, the subjective “strength” of their musical “spark” for that day can be affected by—or can affect—their physiological state at that present moment, and thus would be reflected in their physical voice 105 and/or their physical interaction with a musical instrument to produce sound from it 106. Therefore, this data can be interpreted as biofeedback data. As such, the acoustic-related equipment doesn't have to be actual microphones 103, 104 or a musical instrument 102 capable of sending an audio feed out from it, but any device that is capable of “listening” to the sounds 105, 106 that a user 101 makes, whether from their own physical voice or their physical actions and interactions with a musical instrument 102. Furthermore, audio recognizing/recording devices can be of any size or distance from the user 101, physically connected with the user 101 or simply in the vicinity of the user 101, so long as those audio recognizing/recording devices can properly “listen” to the user 101 and the sounds 105, 106 they can produce. The “Pepper's Ghost”-style system 10B, 10C, 10D can also be of any system or method that simply allows the projected visual data 10F, 10G to appear as if it was “surrounding” and “moving with” the user 101. The screen 109 can also be either a live video feed sent to any receptive device (such as a live internet video feed or a video recording device), or it can be hooked up as part of a musical performance's “light show” where the combined data stream would be displayed on a giant screen behind the user 101. The overhead lighting 10A can either project certain images and/or colors based on the manner of how the computer 108 interprets the audio-based biofeedback data stream of the user's 101 voice 105 and/or instrument playing ability 106.

In this embodiment of the invention, any parameter of the sound may be interpreted as biofeedback variables, to create “artificial synesthesia” for the user. For example, the pitch of the sound may be correlated with different colors, as a simulation of “perfect pitch”. A musician, for example, may wear a wearable computer display and instantly see a color that correlates with the pitch of a sound they are hearing. This would assist the musician in playing along with other musicians or with recorded music. An audience member, too, would find their music listening experience to be enhanced by being able to identify the musical pitch or key of the piece.

In another embodiment, finer distinctions in pitch may be correlated with colors; for example, a musician may use a wearable computer display in helping them tune an instrument by watching for the right color, or in helping them sing in tune.

Other musical parameters may also be used. For example, the visual display may be correlated with the volume of the sound—i.e. getting brighter when the sound gets louder, and getting more muted when the sound gets softer. Different colors may also be correlated with different timbres of sound—i.e. a different color or set of colors for a violin sound than for a piano sound. This will enhance the audience's listening experience.

Other applications of the present invention may also be possible and desirable. For example, a biofeedback sensor may be designed to measure the level of a medication in a patient's bloodstream, and display it as an “aura” when a doctor or nurse looks at the patient. The present invention may also be connected to a pulse oximeter to visually display the patient's oxygen level, a blood sugar sensor to visually display a diabetic patient's blood sugar, or to any other medical sensor to display any sort of medical parameter visually. In another application, the sensor may be a brain wave sensor to measure levels of consciousness in a coma patient, or levels of pain in a chronic pain patient. Multiple sensors may be used as welt for patients with more complex medical needs. In this embodiment of the present invention, the display unit is preferably a portable device such as a smartphone or a wearable display device such as Google Glass. The information may be displayed as a colored “aura”, as text, or even as animations (dancing animated sugar cubes to indicate blood sugar levels, or dancing pink elephants to indicate the levels of a psychiatric medication, alcohol, or recreational drugs in a patient's bloodstream). The advantage of this sort of display is that a doctor can perceive instantly whether or not a patient is in need of help, and that the patient does not even need to verbalize their need (which may help in cases where the patient is unable to speak).

The present invention may also be used as an assistive device for people with disabilities. For example, an autistic person may be unable to perceive a person's mood, interest, or engagement level when communicating with them. A biofeedback sensor can measure all of these things and provide the autistic person with a visual or textual indicator of how interested the other person is in their conversation and what kind of mood the other person is in. As another example, a deaf person may benefit from having the sound of a person's voice displayed visually as an aura around the person, which may enhance lipreading ability and improve communication.

The advantages of the present invention are that it enables biofeedback data to be displayed visually. This may enhance communication by providing instant visual indication of a person's mood or other biofeedback parameters, provide entertainment by providing visual accompaniment to a musical performance, or enhance perception by providing visual indications of parameters that a user is unable to perceive directly—for example, the amount of medication in a patient's bloodstream, the pitch of a musical note (for those without perfect pitch), the mood or interest level of a person (for autistic users), and so on.

In broad embodiment, the present invention is a system and method that allows a computer to record and save data about at least one user's outward physical and inward biological state in real time, and then translate that data into augmented reality form for the user themselves and/or any other interested person(s).

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

1. A system for enhancing a musical performance, comprising:
a sound sensor module for continuously capturing musical sound made by a source of musical sound;
a camera for continuously capturing a real-world scene that includes the source of musical sound;
a computer capable of processing information received from the sound sensor module into visual information, and capable of detecting the location of the user's body in the real-world scene;
a display unit that displays the visual information overlaid on top of the real-world scene in such a way that the location of the visual information depends on the location of the source of musical sound.

2. The system of claim 1, where the display unit is one of the following: a computer screen, a television screen, a smartphone screen, a tablet screen, virtual-reality glasses, image projector, 3D display.

3. The system of claim 1, where the display unit projects the visual information onto a user's body.

4. The system of claim 1, where the sound sensor module gathers data from a musical instrument.

5. The system of claim 1, where the visual information comprises a color field that appears to surround a user's body.

6. The system of claim 1, where the information received from the sound sensor module comprises pitch information.

7. The system of claim 1, where the information received from the sound sensor module comprises timbre information.
